(19) United States
(12) Patent Application Publication
(10) Pub. No.: US 2005/0084457 A1
(43) Pub. Date: Apr. 21, 2005

(54) Capsules containing inhalable tiotropium

(75) Inventors: Dieter Hochrainer, Schmallenberg (DE); Karoline Bechtold-Peters, Biberach-Rissegg (DE); Michael Trunk, Ingelheim am Rhein (DE); Michael Walz, Bingen am Rhein (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma KG, Ingelheim (DE)

(21) Appl. No.: 10/901,790

(22) Filed: Jul. 29, 2004

Related U.S. Application Data
(63) Continuation of application No. 10/159,451, filed on May 31, 2002.
(60) Provisional application No. 60/304,288, filed on Jul. 9, 2001.

(30) Foreign Application Priority Data
Jun. 1, 2001 (DE) 101 26 924

(51) Int. Cl. A61L 9/04; A61K 9/14

(52) U.S. Cl. 424/46; 514/291

(57) ABSTRACT

The invention relates to capsules for inhalation (inhalettes) made from specific capsule materials with a reduced moisture content, which contain the active substance tiotropium in the form of powdered preparations and are characterised by increased stability.

### RELATED APPLICATIONS

Benefit of U.S. Provisional Application Ser. No. 60/304,288, filed on Jul. 9, 2001 is hereby claimed, and said application is herein incorporated by reference in its entirety.

### FIELD OF THE INVENTION

The invention relates to capsules for inhalation (inhalettes) consisting of specific capsule materials with a reduced moisture content, which contain the active substance tiotropium in the form of powdered preparations and are characterised by increased stability.

### BACKGROUND OF THE INVENTION

Tiotropium bromide is known from European Patent Application EP 418 716 A1 and has the following chemical structure:

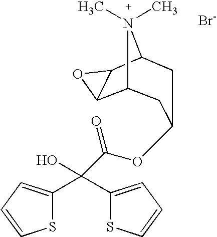

Tiotropium bromide is a highly effective anticholinergic with a long-lasting effect, which may be used to treat respiratory complaints, particularly COPD (chronic obstructive pulmonary disease) and asthma. By tiotropium is meant the free ammonium cation.

When treating the above diseases it is convenient to administer the active substance by inhalation. In addition to the administration by inhalation of broncholytically active compounds in the form of metered aerosols and solutions these medicaments may also be administered in the form of inhalable powders containing active substance.

In the case of active substances with a particularly high efficacy, only small amounts of the active substance are needed per single dose to achieve the therapeutically desired effect. In such cases the active substance has to be diluted with suitable excipients to prepare the inhalable powder. In the case of active substances characterised by a particularly high efficacy it is particularly important, in order to ensure that the content administered remains reproducibly constant, to prepare the pharmaceutical composition in a form which is characterised by a high degree of stability. If this high degree of stability is not achieved, uniform dosage of the active substance cannot be guaranteed.

The aim of the invention is to prepare capsule for inhalation containing an inhalable powder which contains tiotropium, which guarantees sufficient stability of the active substance.

A further aim of the invention is to prepare a capsule for inhalation which by virtue of its stability ensures that the active substance is released with a high metering accuracy (with regard to the amount of active substance and powder mixture packed into each capsule by the manufacturer and also the quantity of active substance released by each capsule in the inhalation process and delivered to the lungs).

The present invention also sets out to prepare a capsule for inhalation which enables the active substance to be administered while emptying the capsule completely.

A further aim of the invention is to prepare capsules for inhalation which have good perforation qualities with good stability and low brittleness and which can therefore be used without any problems in inhalers designed for the administration of inhalettes.

### BRIEF DESCRIPTION OF THE DRAWING

FIG. 1 depicts an example of an inhaler that can be used to administer inhalable powder contained in a capsule for inhalation according to the invention to a patient.

### DETAILED DESCRIPTION OF THE INVENTION

Surprisingly, it has now been found that the problems set out above are solved by the capsules for inhalation (inhalettes) according to the invention described hereinafter.

The capsules for inhalation (inhalettes) according to the invention are capsules which contain, as the inhalable powder, tiotropium mixed with a physiologically acceptable excipient, characterised in that the capsule material has a reduced moisture content.

The concept of a reduced moisture content within the scope of the present invention is defined as being equivalent to a TEWS moisture level of less than 15%.

The term TEWS moisture level within the scope of the present invention means the moisture level which can be determined using the MW 2200 moisture measuring apparatus made by TEWS. The method of measurement used is an indirect one. The activities derived from the water content (microwave absorption by the water contained in the product) are measured and indicated as a microwave value. In order to determine the water content in percent by weight, the apparatus has to be calibrated using calibration samples. The resulting calibration curve is used by the apparatus in subsequent measurements for calculations. The moisture level is given in % and stored in the memory.

A halogen drier, for example, may be used to calibrate the TEWS apparatus within the scope of the present invention. Because the TEWS apparatus is calibrated using a halogen drier within the scope of the present invention the concept of the TEWS moisture level is to be regarded as equivalent to the concept of the halogen drier moisture content. For example, within the scope of the present invention, a 15% TEWS moisture level corresponds to a halogen drier moisture content of 15%. Whereas the TEWS apparatus represents a relative method of measuring water content, by virtue of its mode of operation, the halogen drier gives absolute values for the capsule moisture content. The water content is determined by weight loss in the halogen drier. The capsules are heated, whereupon the water escapes. The capsules are dried until a constant weight is achieved and then the results are read off. The difference in mass between the starting weight and final weight (in grams) represents the water content of the capsules and can be converted into percent by weight. When measuring the water content the TEWS apparatus merely compares the measurement curves of the actual capsules with internal calibration curves. These calibration curves are recorded using capsules with a defined water content whose absolute water content has been determined beforehand using the halogen drier method. In this way, the correlation with absolute water contents is established for the TEWS relative method with the aid of the halogen drier method.

Preferred capsules for inhalation according to the invention have a TEWS or halogen drier moisture content of less than 12%, particularly preferably ≦10%.

By capsule material is meant, within the scope of the present invention, the material from which the shell of the capsule for inhalation is made. The capsule material is according to the invention selected from among gelatine, cellulose derivatives, starch, starch derivatives, chitosan and synthetic plastics.

If gelatine is used as the capsule material, it may be used in admixture with other additives selected from among polyethyleneglycol (PEG), preferably PEG 3350, glycerol, sorbitol, propyleneglycol, PEO-PPO block copolymers and other polyalcohols and polyethers. Within the scope of the present invention gelatine is used particularly preferably in admixture with PEG, preferably PEG 3350. A gelatine capsule according to the invention preferably contains PEG in an amount of 1-10% (wt.-%), preferably 3-8%. Particularly preferred gelatine capsules contain PEG in an amount of 4-6%, a PEG content of about 5% being most preferred according to the invention.

In the case of gelatine-containing capsule materials, the capsules according to the invention preferably have a TEWS or halogen drier moisture content of less than 12%, particularly preferably ≦10%.

If cellulose derivatives are used as the capsule material, it is preferable to use hydroxypropylmethylcellulose, hydroxypropylcellulose, methylcellulose, hydroxymethylcellulose and hydroxyethylcellulose. In this case, hydroxypropylmethylcellulose (HPMC), particularly preferably HPMC 2910 is used as the capsule material. When cellulose derivatives are used as capsule materials the level of the TEWS or halogen drier moisture content is preferably less than 8%, particularly preferably less than 5%. Most preferably, capsules for inhalation consisting of cellulose derivatives are dried to a TEWS or halogen drier moisture content of less than 4%, particularly preferably less than 2%, before being filled with the inhalable powder containing tiotropium.

If synthetic plastics are used as the capsule material, these are preferably selected according to the invention from among polyethylene, polycarbonate, polyester, polypropylene and polyethylene terephthalate. Particularly preferred synthetic plastics for the capsules for inhalation according to the invention are polyethylene, polycarbonate or polyethylene terephthalate. If polyethylene is used as one of the particularly preferred capsule materials according to the invention, polyethylene with a density of between 900 and 1000 kg/³, preferably from 940-980 kg/m³, particularly preferably 960 kg/m³ (high-density polyethylene) is preferably used. When synthetic plastics are used as the capsule materials the level of the TEWS or halogen drier moisture content is optionally less than 3%, optionally less than 1%.

After the empty capsules have been prepared in one of the embodiments mentioned hereinbefore, the capsules for inhalation according to the invention are filled with inhalable powder containing tiotropium. This may be done using methods known from the art. The empty capsules for inhalation to be used for filling may also be prepared using methods known from the prior art. For example, possible production methods include the dipping method, the blast pressure method, injection moulding, extrusion and deep drawing, all of which are known in the art.

When producing the capsules for inhalation according to the invention it is essential that, if the capsule material does not already have a suitably reduced moisture content as a result of its storage or production before being filled with the inhalable powder containing the active substance, the empty capsules are dried. This drying is carried out until a moisture level is reached which corresponds to the specification of not more than 15% TEWS or halogen drier moisture content according to the invention.

Within the scope of the present invention the term capsule for inhalation is to be regarded as synonymous with the word Inhalette.

In another aspect the present invention relates to the use of capsules which are characterised by a TEWS or halogen drier moisture content of less than 15% and may consist of the abovementioned capsule materials, for preparing inhalettes (capsules for inhalation) which contain tiotropium-containing inhalable powder. Within the scope of the present invention the term capsule is to be taken as a reference to empty capsules for inhalation, i.e. ones which do not yet contain any inhalable powder. According to the invention, capsules for inhalation which contain inhalable powder with a tiotropium content of 0.001 to 2% are preferred. Capsules for inhalation which contain inhalable powder with a tiotropium content of 0.04 to 0.8%, preferably 0.08 to 0.64%, particularly preferably 0.16 to 0.4% are particularly preferred. The percentages specified with regard to the content of tiotropium within the scope of the present invention correspond to percent by weight, based on the total quantity of inhalable powder.

By tiotropium is meant the free ammonium cation. The counter-ion (anion) may be chloride, bromide, iodide, methanesulphonate, para-toluenesulphonate or methyl sulphate. Of these anions, the bromide is preferred. Accordingly, the present invention preferably relates to inhalettes containing inhalable powders which are characterised by a tiotropium bromide content of 0.0012-2.41%.

According to the invention, it is particularly advantageous to use inhalable powders which contain between 0.048 and 0.96%, preferably 0.096 to 0.77%, particularly preferably 0.19 to 0.48% tiotropium bromide.

The inhalable powders contained in the inhalettes according to the invention may contain the tiotropium bromide preferably contained therein in the form of its hydrates. Crystalline tiotropium bromide monohydrate is most preferably used. Accordingly the present invention relates to inhalettes which contain powders for inhalation containing between 0.0012 and 2.5% of crystalline tiotropium bromide monohydrate. Of particular interest according to the invention are inhalettes which contain inhalable powders having a crystalline tiotropium bromide monohydrate content of 0.05 to 1%, preferably 0.1 to 0.8%, most preferably 0.2 to 0.5%.

Within the scope of the present invention, any reference to the term tiotropium bromide monohydrate is preferably to be understood as being a reference to the particular crystalline tiotropium bromide monohydrate which can be obtained by the method of synthesis detailed in the experimental section.

The inhalable powders put into the capsules for inhalation (inhalettes) according to the invention contain, in addition to the active substance, at least one excipient. This may consist of an excipient fraction which is uniform in terms of the average particle size of the excipient (e.g. 15-80 μm) or optionally denotes a mixture of coarser excipient with an average particle size of 15 to 80 μm and finer excipient with an average particle size of 1 to 9 μm. If excipient mixtures of coarser and finer excipient fractions are used, the proportion of finer excipient in the total quantity of excipient is preferably 1 to 20%.

Most preferably, if the capsules for inhalation according to the invention consist of a mixture of coarser and finer excipient fractions, they contain coarser excipient with an average particle size of 17 to 50 μm, most preferably 20 to 30 μm, and finer excipient with an average particle size of 2 to 8 μm, most preferably 3 to 7 μm. The phrase average particle size used here denotes the 50% value from the volume distribution measured with a laser diffractometer using the dry dispersion method. Inhalable powders for preparing the inhalettes according to the invention in which the proportion of finer excipient in the total amount of excipient is from 3 to 15%, most preferably 5 to 10%, are preferably used. The percentages given within the scope of the present invention are always percent by weight.

When reference is made to a mixture within the scope of the present invention, this always means a mixture obtained by mixing together clearly defined components. Accordingly, when an excipient mixture of coarser and finer excipients is mentioned, this can only denote mixtures obtained by mixing a coarser excipient component with a finer excipient component.

The excipient fractions may consist of chemically identical or chemically different substances, while inhalable powders in which the excipient fractions consist of the same chemical compound are preferred.

If a mixture of coarser and finer excipient fractions is used as the excipient, these may also consist of chemically identical or chemically different substances, while inhalable powders in which the coarser excipient fraction and the finer excipient fraction consist of the same chemical compound are preferred.

Examples of physiologically acceptable excipients which may be used to prepare the inhalable powders used in the inhalettes according to the invention include, for example, monosaccharides (e.g. glucose or arabinose), disaccharides (e.g. lactose, saccharose, maltose), oligo- and polysaccharides (e.g. dextran), polyalcohols (e.g. sorbitol, mannitol, xylitol), salts (e.g. sodium chloride, calcium carbonate) or mixtures of these excipients with one another. Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred.

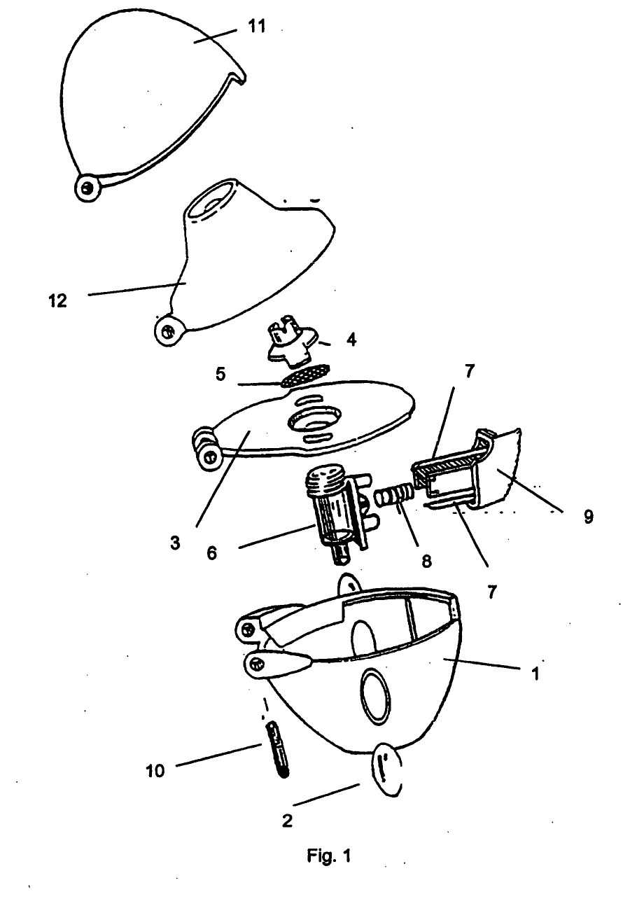

The capsules for inhalation according to the invention may, for example, be administered using inhalers as described in WO 94/28958. A particularly preferred inhaler for using the inhalettes according to the invention is shown in exploded view in FIG. 1.

This inhaler (Handihaler) for inhaling powdered pharmaceutical compositions from capsules is characterised by a housing 1 containing two windows 2, a deck 3 in which there are air inlet ports and which is connected to the capsule chamber 6, on which is provided a push button 9 provided with two sharpened pins 7 and movable counter to a spring 8, and a mouthpiece 12 which is connected to the housing 1, the deck 3 and a cover 11 via a spindle 10 to enable it to be flipped open or shut. The capsule chamber is closed off by a filter 5. The filter is carried by a filter holder fixed to the mouthpiece 12.

The capsules for inhalation according to the invention may contain amounts of from 2 to 50 mg, preferably 4 to 25 mg of inhalable powder per capsule for inhalation. They then contain between 1.2 and 80 μg of tiotropium. If each capsule for inhalation contains the particularly preferred amount of 4 to 6 mg of inhalable powder, each one contains between 1.6 and 48 μg, preferably between 3.2 and 38.4 μg, particularly preferably between 6.4 and 24 μg of tiotropium. A tiotropium content of 18 μg for example corresponds to a tiotropium bromide content of about 21.7 μg.

Consequently, capsules for inhalation containing 3 to 10 mg of inhalable powder according to the invention preferably contain between 1.4 and 96.3 μg of tiotropium bromide. With a preferred content of 4 to 6 mg of inhalable powder in each capsule for inhalation, the content of tiotropium bromide in each capsule is between 1.9 and 57.8 μg, preferably between 3.9 and 46.2 μg, particularly preferably between 7.7 and 28.9 μg. A tiotropium bromide content of 21.7 μg of tiotropium bromide, for example, corresponds to a tiotropium bromide monohydrate content of about 22.5 μg.

Consequently, capsules for inhalation containing 3 to 10 mg of inhalable powder preferably contain between 1.5 and 100 μg of tiotropium bromide-monohydrate. With a preferred content of 4 to 6 mg of inhalable powder in each capsule for inhalation, the tiotropium bromide monohydrate content of each capsule is between 2 and 60 μg, preferably between 4 and 48 μg, particularly preferably between 8 and 30 μg.

The capsules for inhalation according to the invention are characterised in accordance with the objective of the present invention by a high degree of homogeneity in terms of the accuracy of single doses. This accuracy is in the region of <8%, preferably <6%, particularly preferably <4%.

The inhalable powders preferably used to fill the capsules for inhalation according to the invention may be obtained by the method described below.

After the starting materials have been weighed out, the next step is to prepare the mixture of excipients, in those cases where the excipient used is a mixture of coarser and finer fractions. If the excipient used is a uniform fraction in terms of its average particle size (e.g. 15-80 μm), this first step can be omitted.

The inhalable powder is then prepared from the excipient, possibly the mixture of excipients, and the active substance. The capsules for inhalation according to the invention are dried before filling with the tiotropium-containing inhalable powder until the maximum permissible level of TEWS or halogen drier moisture content according to the invention is reached. Then the powder-filled capsules for inhalation are produced using methods known in the art.

In the preparation processes described hereinafter, the abovementioned components are used in the amounts by weight described in the abovementioned compositions of the inhalable powders.

If mixtures of coarser and finer excipient fractions are used as the excipient, the coarser and finer excipient fractions are placed in a suitable mixing container. The two components are preferably added using a granulating sieve with a mesh size of 0.1 to 2 mm, preferably 0.3 to 1 mm, most preferably 0.3 to 0.6 mm. Preferably, the coarser excipient is put in first and then the finer excipient fraction is added to the mixing container. During this mixing process the two components are preferably added in batches, with some of the coarser excipient being put in first and then finer and coarser excipient being added alternately. It is particularly preferred when producing the excipient mixture to sieve in the two components in alternate layers. The two components are preferably sieved in alternately in 15 to 45, most preferably 20 to 40 layers each. The mixing of the two excipients may take place while the two components are still being added. Preferably, however, mixing is only done once the two components have been sieved in layer by layer.

This step is, of course, omitted if an excipient fraction of uniform particle size is used (e.g. average particle size of 15-80 μm).

Then the excipient, optionally the excipient mixture and the active substance are placed in a suitable mixing container. The active substance used has an average particle size of 0.5 to 10 μm, preferably 1 to 6 μm, most preferably 2 to 5 μm. The two components are preferably added using a granulating sieve with a mesh size of 0.1 to 2 mm, preferably 0.3 to 1 mm, most preferably 0.3 to 0.6 mm. Preferably, the excipient mixture is put in first and then the active substance is added to the mixing container. During this mixing process the two components are preferably added in batches. It is particularly preferred when producing the excipient mixture to sieve in the two components in alternate layers. The two components are preferably sieved in alternately in 25 to 65, most preferably 30 to 60 layers. The mixing of the excipient mixture with the active substance may take place while the two components are still being added. Preferably, however, mixing is only done once the two components have been sieved in layer by layer. The powder mixture thus obtained may optionally be added once or repeatedly using a granulating sieve and then subjected to another mixing process.

In another preferred embodiment of the invention, the capsules for inhalation are filled with the inhalable powder containing tiotropium bromide obtained by the above process and then subjected to the dryness process described as follows.

The filled capsules are subjected in a first phase (drying phase) for a period of 0.5-10 hours, preferably 1.5-7 hours, preferably 2-5.5 hours, particularly preferably about 2.5-4.5 hours at a temperature of about 10-50° C., preferably 20-40° C., particularly preferably about 25-35° C. to a relative humidity of not more than 30% r.h., preferably not more than 20% r.h., particularly preferably about 5-15% r.h. By relative humidity (r.h.) is meant, within the scope of the present invention, the quotient of the partial steam pressure and the steam pressure at the temperature in question. In a subsequent second phase (equilibrium phase) the capsules are exposed to a relative humidity of not more than 35% r.h., preferably not more than 25% r.h., particularly preferably about 10-20% r.h. for a period of 0.5-10 hours, preferably 1.5-7 hours, preferably 2-5.5 hours, particularly preferably about 2.5-4.5 hours at a temperature of about 10-50° C., preferably 20-40° C., particularly preferably about 25-35° C. This is optionally followed by a cooling phase if the temperature in the preceding steps was adjusted to levels above ambient temperature (i.e. 23° C.). During this cooling phase the capsules are exposed to a relative humidity of at most 35% r.h., preferably at most 25% r.h., particularly preferably about 10-20% r.h. for a period of 0.1-6 hours, preferably 0.5-4 hours, preferably 0.75-2.5 hours, particularly preferably about 1-2 hours at a temperature of about 23° C. In a particularly preferred embodiment the values set for the relative humidity in the equilibrium phase and cooling phase are identical.

When the term active substance is used within the scope of the present invention, this is intended as a reference to tiotropium. According to the invention, any reference to tiotropium, which is the free ammonium cation, corresponds to a reference to tiotropium in the form of a salt (tiotropium salt) which contains an anion as the counter-ion. Tiotropium salts which may be used within the scope of the present invention are those compounds which contain chloride, bromide, iodide, methanesulphonate, para-toluenesulphonate or methyl sulphate, in addition to tiotropium as counter-ion (anion). Within the scope of the present invention, tiotropium bromide is preferred of all the tiotropium salts. References to tiotropium bromide within the scope of the present invention should always be taken as references to all possible amorphous and crystalline modifications of tiotropium bromide. These may, for example, include molecules of solvent in their crystalline structure. Of all the crystalline modifications of tiotropium bromide, those which also include water (hydrates) are preferred according to the invention. It is particularly preferable to use tiotropium bromide monohydrate within the scope of the present invention, which may be obtained using the procedure described in detail hereinafter.

In order to prepare the capsules for inhalation containing tiotropium according to the invention, first of all tiotropium has to be prepared in a form which can be used for pharmaceutical purposes. For this, tiotropium bromide, which may be prepared as disclosed in EP 418 716 A1, is preferably subjected to another crystallisation step. Depending on the reaction conditions and solvent used, different crystal modifications are obtained. For the purposes of preparing the capsules for inhalation according to the invention it has proved particularly suitable to use crystalline tiotropium bromide monohydrate.

The following Examples serve to illustrate the present invention in more detail without restricting the scope of the invention to the embodiments described hereinafter by way of example.

Starting Materials

In the Examples which follow, lactose-monohydrate (200M-) is used as the excipient. It may be obtained, for example, from Messrs DMV International, 5460 Veghel/NL under the product name Pharmatose 200M.

In the Examples which follow, lactose-monohydrate (200M) is also used as the coarser excipient when excipient mixtures are used. It may be obtained, for example, from Messrs DMV International, 5460 Veghel/NL under the product name Pharmatose 200M.

In the Examples which follow, when excipient mixtures are used, lactose-monohydrate (5 μm) is used as the finer excipient. It may be obtained from lactose-monohydrate 200M by conventional methods (micronising). Lactose-monohydrate 200M may be obtained, for example, from Messrs DMV International, 5460 Veghel/NL under the product name Pharmatose 200M.

Preparation of Crystalline Tiotropium Bromide Monohydrate:

15.0 kg of tiotropium bromide are added to 25.7 kg of water in a suitable reaction vessel. The mixture is heated to 80-90° C. and stirred at constant temperature until a clear solution is formed. Activated charcoal (0.8 kg), moistened with water, is suspended in 4.4 kg of water, this mixture is added to the solution containing the tiotropium bromide and rinsed with 4.3 kg of water. The mixture thus obtained is stirred for at least 15 min at 80-90° C. and then filtered through a heated filter into an apparatus which has been preheated to an outer temperature of 70° C. The filter is rinsed with 8.6 kg of water. The contents of the apparatus are cooled by 3-5° C. every 20 minutes to a temperature of 20-25° C. The apparatus is further cooled to 10-15° C. using cold water and crystallisation is completed by stirring for at least one hour. The crystals are isolated using a suction drier, the crystal slurry isolated is washed with 9 litres of cold water (10-15° C.) and cold acetone (10-15° C.). The crystals obtained are dried in a nitrogen current at 25° C. over 2 hours.

Yield: 13.4 kg of tiotropium bromide monohydrate (86% of theory)

The crystalline tiotropium bromide monohydrate obtainable using the method described above was investigated by DSC (Differential Scanning Calorimetry). The DSC diagram shows two characteristic signals. The first, relatively broad, endothermic signal between 50-120° C. can be attributed to the dehydration of the tiotropium bromide monohydrate into the anhydrous form. The second, relatively sharp, endothermic peak at 230±5° C. can be put down to the melting of the substance. This data was obtained using a Mettler DSC 821 and evaluated using the Mettler STAR software package. The data was recorded at a heating rate of 10 K/min.

The crystalline tiotropium bromide monohydrate according to the invention was characterised by IR spectroscopy. The data was obtained using a Nicolet FTIR spectrometer and evaluated with the Nicolet OMNIC software package, version 3.1. The measurement was carried out with 2.5 μmol of tiotropium bromide monohydrate in 300 mg of KBr.

The following Table shows some of the essential bands of the IR spectrum.

| Wave number (cm⁻¹) | Attribution | Type of oscillation |
|---|---|---|
| 3570, 410 | O—H | elongated oscillation |
| 3105 | Aryl C—H | elongated oscillation |
| 1730 | C═O | elongated oscillation |
| 1260 | Epoxide C—O | elongated oscillation |
| 1035 | Ester C—OC | elongated oscillation |
| 720 | Thiophene | cyclic oscillation |

The crystalline tiotropium bromide monohydrate obtainable by the above process has, according to single crystal X-ray structural analysis, a simple monoclinic cell with the following dimensions: a=18.0774 Å, b=11.9711 Å, c=9.9321 Å, □=102.691°, V=2096.96 Å³. These data were collected on an AFC7R-4-circuit diffractometer (Rigaku) using monochromatic copper K_{□} radiation. The structural solution and refinement of the crystal structure were obtained by direct methods (SHELXS86 Program) and FMLQ-refinement (TeXsan Program).

The crystalline tiotropium bromide monohydrate thus obtained is micronised by known methods to prepare the active substance in the form of the average particle size corresponding to the specifications according to the invention.

The method of determining the average particle size of the various ingredients of the formulation according to the invention is described as follows.

A) Determining the Particle Size of Finely Divided Lactose:

Measuring Equipment and Settings:

The equipment is operated according to the manufacturer's instructions.

| | |
|---|---|
| Measuring equipment: | HELOS Laser-diffraction |
| | spectrometer, (SympaTec) |
| Dispersing unit: | RODOS dry disperser with |
| | suction funnel, (SympaTec) |
| Sample quantity: | from 100 mg |
| Product feed: | Vibri Vibrating channel, |
| | Messrs. Sympatec |
| Frequency of vibrating | 40 rising to 100% |
| channel: | |
| Duration of sample feed: | 1 to 15 sec. (in the case |
| | of 100 mg) |
| Focal length: | 100 mm (measuring |
| | range: 0.9-175 μm) |
| Measuring time: | about 15 s (in the case of 100 mg) |
| Cycle time: | 20 ms |
| Start/stop at: | 1% on channel 28 |
| Dispersing gas: | compressed air |
| Pressure: | 3 bar |
| Vacuum: | maximum |
| Evaluation method: | HRLD |

Sample Preparation/Product Feed:

At least 100 mg of the test substance are weighed onto a piece of card.

Using another piece of card all the larger lumps are broken up. The powder is then sprinkled finely over the front half of the vibrating channel (starting about 1 cm from the front edge). After the start of the measurement the frequency of the vibrating channel is varied from about 40% up to 100% (towards the end of the measurement). The time taken to feed in the entire sample is 10 to 15 sec.

B) Determining the Particle Size of Micronised Tiotropium Bromide Monohydrate:

Measuring Equipment and Settings:

The equipment is operated according to the manufacturer's instructions.

| | |
|---|---|
| Measuring equipment: | Laser diffraction spectrometer |
| | (HELOS), Sympatec |
| Dispersing unit: | RODOS dry disperser with suction f [symbol] |
| Sample quantity: | 50 mg-400 mg |
| Product feed: | Vibri Vibrating channel, |
| | Messrs. Sympatec |
| Frequency of vibrating | 40 rising to 100% |
| channel: | |
| Duration of sample feed: | 15 to 25 sec. (in the case of 200 mg) |
| Focal length: | 100 mm (measuring range: 0.9-175 μm) |
| Measuring time: | about 15 s (in the case of 200 mg) |
| Cycle time: | 20 ms |
| Start/stop at: | 1% on channel 28 |
| Dispersing gas: | compressed air |
| Pressure: | 3 bar |
| Vacuum: | maximum |
| Evaluation method: | HRLD |

Sample Preparation/Product Feed:

About 200 mg of the test substance are weighed onto a piece of card.

Using another piece of card all the larger lumps are broken up. The powder is then sprinkled finely over the front half of the vibrating channel (starting about 1 cm from the front edge). After the start of the measurement the frequency of the vibrating channel is varied from about 40% up to 100% (towards the end of the measurement). The sample should be fed in as continuously as possible. However, the amount of product should not be so great that adequate dispersion cannot be achieved. The time over which the entire sample is fed in is about 15 to 25 seconds for 200 mg, for example.

C) Determining the Particle Size of Lactose 200M:

Measuring Equipment and Settings:

The equipment is operated according to the manufacturer's instructions.

| | |
|---|---|
| Measuring equipment: | Laser diffraction spectrometer |
| | (HELOS), Sympatec |
| Dispersing unit: | RODOS dry disperser with |
| | suction funnel, Sympatec |
| Sample quantity: | 500 mg |
| Product feed: | VIBRI Vibrating channel, |
| | Messrs. Sympatec |
| Frequency of vibrating | 18 rising to 100% |
| channel: | |
| Focal length (1): | 200 mm (measuring range: 1.8-350 μm) |
| Focal length (2): | 500 mm (measuring range: 4.5-875 μm) |
| Measuring time: | 10 s |
| Cycle time: | 10 ms |
| Start/stop at: | 1% on channel 19 |
| Pressure: | 3 bar |
| Vacuum: | maximum |
| Evaluation method: | HRLD |

Sample Preparation/Product Feed:

About 500 mg of the test substance are weighed onto a piece of card.

Using another piece of card all the larger lumps are broken up. The powder is then transferred into the funnel of the vibrating channel. A gap of 1.2 to 1.4 mm is set between the vibrating channel and funnel. After the start of the measurement the amplitude setting of the vibrating channel is increased from 0 to 40% until a continuous flow of product is obtained. Then it is reduced to an amplitude of about 18%. Towards the end of the measurement the amplitude is increased to 100%.

Apparatus

The following machines and equipment, for example, may be used to prepare the inhalable powders:
Mixing container or powder mixer: Gyrowheel mixer 200 L; type: DFW80N-4; made by: Messrs Engelsmann, D-67059 Ludwigshafen.
Granulating sieve: Quadro Comil; type: 197-S; made by: Messrs Joisten & Kettenbaum, D-51429 Bergisch-Gladbach.

To determine the TEWS moisture level the following apparatus is used in accordance with the manufacturer's instructions.

Apparatus for Determining the TEWS Moisture Level:

Manufacturer: Messrs TEWS Elektronik, Hamburg

| | |
|---|---|
| Type: | MW 2200 |
| Measuring range: | 1 to 85% moisture |
| Accuracy of measurement: | 1% of the final value of the |
| | measuring range chosen. |
| Mains connection: | 220 V +/− 10%, 50-60 Hz |

In order to determine the halogen drier moisture content as well as to adapt the TEWS apparatus, the following apparatus is used in accordance with the manufacturer's instructions.

Apparatus for Determining the Halogen Drier Moisture Level:

Mettler halogen drier HR 73; manufacturer: Messrs Mettler-Toledo, D-35396 GieBen;

The following apparatus is used to fill the empty capsules with powder for inhalation containing tiotropium.

Capsule Filling Machine:

MG2, Type G100, manufacturer: MG2 S.r.l, I-40065 Pian di Macina di Pianoro (BO), Italy.

### EXAMPLE 1

1.1: Excipient Mixture:

31.82 kg of lactose monohydrate for inhalation (200M) are used as the coarser excipient component. 1.68 kg of lactose monohydrate (5 μm) are used as the finer excipient component. In the resulting 33.5 kg of excipient mixture the proportion of the finer excipient component is 5%.

About 0.8 to 1.2 kg of lactose monohydrate for inhalation (200M) are added to a suitable mixing container through a suitable granulating sieve with a mesh size of 0.5 mm. Then alternate layers of lactose monohydrate (5 μm) in batches of about 0.05 to 0.07 kg and lactose monohydrate for inhalation (200M) in batches of 0.8 to 1.2 kg are sieved in. Lactose monohydrate for inhalation (200M) and lactose monohydrate (5 μm) are added in 31 and 30 layers, respectively (tolerance: ±6 layers).

The ingredients sieved in are then mixed together (mixing at 900 rpm).

1.2: Final Mixture:

To prepare the final mixture, 32.87 kg of the excipient mixture (1.1) and 0.13 kg of micronised tiotropium bromide monohydrate are used. The content of active substance in the resulting 33.0 kg of inhalable powder is 0.4%.

The same procedure is followed using only 32.87 kg of lactose monohydrate (200 M) if an excipient fraction of uniform average particle size is used as the excipient. In this case, step 1.1 is naturally omitted.

About 1.1 to 1.7 kg of excipient or excipient mixture (1.1) are added to a suitable mixing container through a suitable granulating sieve with a mesh size of 0.5 mm. Then alternate layers of tiotropium bromide monohydrate in batches of about 0.003 kg and excipient or excipient mixture (1.1) in batches of 0.6 to 0.8 kg are sieved in. The excipient or excipient mixture and the active substance are added in 47 or 45 layers, respectively (tolerance: ±9 layers).

The ingredients sieved in are then mixed together (mixing at 900 rpm). The final mixture is passed through a granulating sieve twice more and then mixed (mixing at 900 rpm).

### EXAMPLE 2

Inhalation capsules (inhalettes) having the following composition were produced using the mixture obtained according to Example 1:

| | | |
|---|---|---|
| tiotropium bromide monohydrate: | 0.0225 | mg |
| lactose monohydrate (200 M): | 5.2025 | mg |
| lactose monohydrate (5 μm): | 0.2750 | mg |
| hard gelatine capsule (5% PEG 3350; 9% TEWS moisture): | 49.0 | mg |
| Total: | 54.5 | mg |

### EXAMPLE 3

Inhalation Capsules:

| | | |
|---|---|---|
| tiotropium bromide monohydrate: | 0.0225 | mg |
| lactose monohydrate (200 M): | 4.9275 | mg |
| lactose monohydrate (5 μm): | 0.5500 | mg |
| hard gelatine capsule (5% PEG 3350; 9% TEWS moisture): | 49.0 | mg |
| Total: | 54.5 | mg |

The inhalable powder needed to prepare the capsules was obtained analogously to Example 1.

### EXAMPLE 4

Inhalation Capsules:

| | | |
|---|---|---|
| tiotropium bromide monohydrate: | 0.0225 | mg |
| lactose monohydrate (200 M): | 5.2025 | mg |
| lactose monohydrate (5 μm): | 0.2750 | mg |
| HPMC (<2% TEWS moisture): | 49.0 | mg |
| Total: | 54.5 | mg |

The inhalable powder needed to prepare the capsules was obtained analogously to Example 1.

### EXAMPLE 5

Inhalation Capsules:

| | | |
|---|---|---|
| tiotropium bromide monohydrate: | 0.0225 | mg |
| lactose monohydrate (200 M): | 5.2025 | mg |
| lactose monohydrate (5 μm): | 0.2750 | mg |
| polyethylene (<1% TEWS moisture): | 100.0 | mg |
| Total: | 105.5 | mg |

The inhalable powder needed to prepare the capsules was obtained analogously to Example 1.

### EXAMPLE 6

Inhalation Capsules:

| | | |
|---|---|---|
| tiotropium bromide monohydrate: | 0.0225 | mg |
| lactose monohydrate (200 M): | 5.4775 | mg |
| polyethylene (<1% TEWS moisture): | 100.0 | mg |
| Total: | 105.5 | mg |

The inhalable powder needed to prepare the capsules was obtained analogously to Example 1.

### EXAMPLE 7

Inhalation capsules (inhalettes) having the following composition were produced using the mixture obtained according to Example 1:

| | |
|---|---|
| tiotropium bromide monohydrate: | 0.0225 mg |
| lactose monohydrate (200 M): | 5.2025 mg |
| lactose monohydrate (5 μm): | 0.2750 mg |
| hard gelatine capsule (5% PEG 3350): | 49.0 mg |
| Total: | 54.5 mg |

These capsules are adjusted to a water content of about 8.7% (measured with a TEWS microwave moisture measuring apparatus) under suitable climatic conditions in an air-conditioned chamber using the following procedure.

To start with, a drying phase is carried out, followed by a so-called equilibrium phase. Finally, the capsules are subjected to a so-called cooling phase. The capsules thus dried are then packaged directly afterwards in corresponding storable packaging or the like.

Process Data

Setting the climatic conditions to the following rated values:

| | |
|---|---|
| Drying phase: | 30° C. |
| | 10% r.h. |
| | (relative humidity) |
| | 3.5 h |
| Equilibrium phase: | 30° C. |
| | 16% r.h. |
| | 3.5 h |
| Cooling phase: | 23° C. |
| | 16% r.h. |
| | 1.5 h |

By relative humidity (r.h.) within the scope of the present invention is meant the quotient of the partial steam pressure and the vapour pressure of the water at the temperature in question.

For the purposes of the present invention, the average particle size means the value in μm at which 50% of the particles of the volume distribution have a particle size which is smaller than or the same as the value specified. The laser diffraction/dry dispersion method is used as the method of measuring the total distribution of the particle size distribution.

1) A capsule for inhalation which contains as an inhalable powder a mixture of tiotropium with at least one physiologically acceptable excipient, wherein the material forming the capsule is a mixture of cellulose derivatives, starch, starch derivatives, chitosan and synthetic plastics and has a TEWS or halogen drier moisture of content of less than 15%.

2) (canceled)

3) (canceled)

4) (canceled)

5) (canceled)

6) (canceled)

7) (canceled)

8) (canceled)

9) (canceled)

10) (canceled)

11) (canceled)

12) A capsule for inhalation according to claim 1, wherein the material forming the capsule is selected from hydroxypropylmethylcellulose, hydroxypropylcellulose, methylcellulose, hydroxymethylcellulose and hydroxyethylcellulose.

13) A capsule for inhalation according to claim 12, wherein the material forming the capsule has a TEWS or halogen drier moisture content of less than 8%.

14) A capsule for inhalation according to claim 12, wherein the material forming the capsule has a TEWS or halogen drier moisture content of ≦5%.

15) A capsule for inhalation according to claim 1, wherein the material forming the capsule is selected from polyethylene, polycarbonate, polyester, polypropylene and polyethylene terephthalate.

16) A capsule for inhalation according to claim 15, wherein the material forming the capsule is selected from polyethylene, polycarbonate and polyethylene terephthalate.

17) A capsule for inhalation according to claim 15, wherein the material forming the capsule has a TEWS or halogen drier moisture content of less than 3%.

18) A capsule for inhalation according to claim 15, wherein the material forming the capsule has a TEWS or halogen drier moisture content of ≦1%.

19) A capsule for inhalation according to claim 16, wherein the material forming the capsule has a TEWS or halogen drier moisture content of less than 3%.

20) A capsule for inhalation according to claim 16, wherein the material forming the capsule has a TEWS or halogen drier moisture content of ≦1%.

21) A capsule for inhalation according to claim 1, wherein the inhalable powder contains a mixture of at least one physiologically acceptable excipient with 0.001 to 2% tiotropium.

22) A capsule for inhalation according to claim 21, wherein the excipient consists of a mixture of coarser excipient with an average particle size of 15 to 80 μm and finer excipient with an average particle size of 1 to 9 μm, the proportion of finer excipient in the total quantity of excipient being 1 to 20%.

23) A capsule for inhalation according to claim 22, wherein the tiotropium is in the form of its chloride, bromide, iodide, methanesulphonate, para-toluenesulphonate or methylsulphate.

24) An inhaler suitable for administering inhalable powders containing a capsule for inhalation according to claim 1.

25) A method of treating asthma or COPD in a patient in need thereof comprising administering to said patient an inhalable powder using an inhaler according to claim 24.

26) A method of making a capsule for inhalation according to claim 1 comprising filling an empty capsule having a TEWS or halogen drier moisture content of less than 15% with a mixture of tiotropium with at least one physiologically acceptable excipient.

27) The capsule according to claim 1 wherein the material forming the capsule is selected from starch, starch derivatives and chitosan.

28) The capsule according to claim 1 wherein the material forming the capsule comprises cellulose derivatives having a moisture content in an amount of less that 2% by weight.

29) The capsule according to claim 12 having a moisture content of less than 2% by weight.
